(19) United States
(12) Patent Application Publication
(10) Pub. No.: US 2012/0015361 A1
(43) Pub. Date: Jan. 19, 2012

(54) Oligonucleotides For The Detection Of Aspergillus Species

(76) Inventors: Sándor Bíró, Debrecen (HU); Zsuzsanna Birkó, Debrecen (HU); Melinda Paholcsek, Cegledbercel (HU)

(21) Appl. No.: 13/116,235

(22) Filed: May 26, 2011

Related U.S. Application Data
(63) Continuation-in-part of application No. PCT/IB2009/055369, filed on Nov. 26, 2009.

(30) Foreign Application Priority Data
Nov. 26, 2008 (HU) P0800722

(51) Int. Cl. C12Q 1/68; C07H 21/04

(52) U.S. Cl. 435/6.12; 536/24.32; 435/6.15

(57) ABSTRACT

The invention relates to oligonucleotides that are specific for the fungi belonging to Aspergillus genus, and which are able to hybridize to facC gene homologues present in those fungi and show homology with the facC gene of Streptomyces griseus 45H, the sequence of said homologous gene is identical with any sequences of SEQ ID NO: 118 to 120. These oligonucleotides make possible the detection and identification the members of the Aspergillus genus, specifically the Aspergillus fumigatus or Aspergillus terreus.

### RELATED APPLICATIONS AND INCORPORATION BY REFERENCE

This application is a continuation-in-part application of international patent application Serial No. PCT/IB2009/055369 filed Nov. 26, 2009, which published as PCT Publication No. WO/2010/061351 on Jun. 3, 2010, which claims benefit of Hungarian patent application Serial No. P0800722 filed Nov. 26, 2008.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The field of the invention generally is diagnostic microbiology, particularly the species specific detection and identification of Aspergillus species.

The invention relates to oligonucleotides that are specific for the fungi belonging to Aspergillus genus, and which are able to hybridize to facC gene homologues present in those fungi and show homology with the facC gene of Streptomyces griseus 45H, the sequence of said homologous gene is identical with any sequences of SEQ ID NO: 118 to 120. These oligonucleotides make possible the detection and identification the members of the Aspergillus genus, specifically the Aspergillus fumigatus or Aspergillus terreus.

### BACKGROUND OF THE INVENTION

The saprophytic Aspergillus species are ubiquitous in our environment, however as opportunistic pathogens they only cause systemic diseases/infections in immunocompromised hosts/patients (those with AIDS, acute leukemia, those under intensive cytotoxic chemotherapies). Despite of this besides the Candida species Aspergillus species are the second most common causative agents of nosocomial fungal systemic infections with the incidence of 1/20 000. The explanation of this phenomenon can be found in the changes of the state of the art in the last quarter century, because artificial immunosuppressive treatments drastically increased the number of invasive mycoses.

Despite of the rapid development in antifungal therapy during the past decade, the aspergillosis cases remain a major cause of the infection-related morbidity and mortality in developed countries.

Besides the Candida species the mayor causative agents of the highly devastating systematic mycoses are mainly caused by the filamentous fungi of the Aspergillus genus, such as Aspergillus fumigatus, A. terreus, A. flavus, A. niger and A. nidulans (Pagano et al., 2006). Numerous articles confirm that other human pathogens like Neosartorya (Guano et al., 2002) and Chaetomium (Anandi et al., 1989, Abbott et al., 1995; Yeghen et al., 1996) also play important role in the development of said disease.

Aspergillus species rarely cause disease in healthy persons and these infections cannot disperse among people either. Conidia enters the body by inhalation since the Aspergillus genus relating filamentous fungi are ubiquitous in our environment. They lead a saprophytic life in the soil. Besides infections caused by fungi spores originated from rotten organic residues (compost pitches) and Aspergillus species consumed with pepper, coffee or peanut, nosocomial infections are also important, e.g. infections arising during hospital treatment (especially distributed by the air conditioning apparatuses of the intensive car or other departments) (Vonberg, 2006).

Due to the limited or total immunocompromised state of the individuals these infections may become invasive and in spite of the fact that in the status of the primary disease a distinct improvement is showed, the secondary evolved infections may lead to death.

The infection may become systemic due to the immune defect. After a given time (in months or in years) the infection becomes systemic and through the blood system disseminates in the body e.g. into the central nervous system, liver or kidneys (Vidal et al., 2005; Hummel et al., 2006).

Depending on where the conidia are able to colonize we classify aspergillosis in three main categories. Aspergilloma or mycetoma, allergic bronchopulmonary aspergillosis or aspergilloma of the lungs and finally the invasive aspergillosis, which last one is deadly in almost 100% of the cases.

Survival depends on early started antifungal therapy.

The prevention would be of great significance in case of those patients that belong to the risk group. In their cases regular cost effective screening would be important. The prophylactic use of antimycotics may be able to decrease the frequency of the disease.

Furthermore the species level identification of Aspergilluses is also of great importance, since it is a prerequisite of the targeted antifungal therapy because different species response differently to a given antifungal treatment, not to mention that this way the spread of resistant fungi species may be controlled and decreased. For example Aspergillus terreus is known to be resistant to Amphotericin B, which is among the first line options in antifungal therapy and which lately is combined with different Echinocandins, like with Voriconazol (Segal et al., 2006).

The reliable diagnosis is hampered by some difficulties since the symptoms are not specific and the causative agents of mycosis are hard to identify due to the presence of other causative and concomitant microbes. Furthermore very important is the species level detection, which is the prerequisite of the targeted antifungal therapy.

The most reliable confirmation of the presence of fungus in the attacked tissues is only possible by analyzing or histologically examining fungi cultures originating from appropriate samples, however in most of the cases these procedures are mainly post mortem, on the other hand these cultivating tests are rarely appropriate for species level detection.

Microbiological and histopathological methods are time consuming and they often need samples from biopsies that are not always appropriate due to the risk associated with the disease, because in many patients the biopsy itself is risky.

Different imaging procedures like X-ray, CT, MRI examinations and the results of cultivations from the sputum, nose phlegm, BAL (broncoalveolar lavage) facilitate the diagnosis only in far-gone statement (White et al., 2006a; Erjavec, Verweij, 2002).

Nowadays besides the imaging procedures and culture based confirmations commercially available Aspergillus diagnostic methods can be:
serological procedures/assays like the latex agglutination and ELISA (Enzyme-Linked Immunosorbent Assay) or the sandwich-ELISA, which is the improved form of the previous. In case of these assays the antigen is the in the plasma circulating fungi cell wall component, the (1→5)-β-galactofuranosil side chain of (1→3)-β-D-glucan molecule or other termo stable polysaccharide component like the galactomannan, which consists of a ‘non-immunogen’, called “mannan core” central core, and contains immunoactive side chains (a ligand használható), like galactofuranosil units.
DNA based and the combination of these (Florent et al., 2006; Denning, 2006; Williamson et al., 2000). The advantage of the latter hybrid method is that it is able to combine the high rate (94-100%) specificity of PCR reactions with the high rate (85-100%) sensitivity characteristic in serological methods (Aquino et al., 2007).

The greatest advantage of the serological methods is the rapid applicability. Opposite to the culture based methods these can show results within 3 hours (Aquino et al., 2007), however since these methods screen for ubiquitously present fungal cell wall components, they are not capable for species level detection.

Therefore it is needed to combine these methods with other DNA based detection methods (nested PCR, quantitative-real time-PCR). These are mainly based on the conserved sequences of ribosomal RNA genes (see e.g: EP 979312).

The DNA based methods are highly common because they are fast, easily reproducible and well applicable. Depending on the attributes of the target gene these are able to show high specificity (almost 100%) (Aquino et al, 2007).

Other factors such as the origin of the specimens (blood, BAL-fluid), the efficiency of the DNA isolation, the structure and specificity of the primers (are they able to build primer dimers or other non specific amplified fragments), the type of the PCR reaction (two step, nested, seminested, quantitative-real time-PCR) influences the sensitivity to a great extent.

If fungal fragments can be found in biological samples then they can be detected after the appropriate elaboration in less than one day. Recently the methods based on quantitative real time PCR (Q-RT-PCR) systems are the most widely used (Bolehovska et al., 2006; White et al., 2006/b). The Q-RT-PCR methods are capable of monitoring the amplification procedure cycle by cycle. The quantitative detection of the amplified fragments takes place by measuring fluorescence signals. Another great advantage of the method is that the assays are handled in separated and closed tubes which reduce the possibility of environmental contamination during the processing.

Opposite to the serological methods the appropriately performed assays can identify Aspergilli on species level (Erjavec, Verweij, 2002).

In the case of serological methods a great percentage (14%) of false positive results occur in the case of a patient group who were treated right before the diagnosis by the combination of beta lactam antibiotics and beta lactamase inhibitors like PIPERACILLIN-TAZOBACTAM, AMPICILLIN-SULBACTAM and AMOXICILLIN-CLAVULANIC ACID or with other antibiotics such as Penicillin-G, Ceftriaxon, Imipenem, Ciprofloxacin, Vancomocin, Gentamicin and so on (Aquino et al., 2007).

These cases can be explained by artificial contamination. Artificial contamination can happen accidentally during sample preparation in the case of a healthy patient and in patients under antibiotics treatment artificial contaminants are introduced into the bloodstream, in the cell wall of beta lactam antibiotics producing Acremonium genus galactofuranosil ligands can be found as well (Florent et al., 2006).

In case of real time PCR system processed reactions based on the detection of specific ribosomal RNA genes that are present generally in fungi it is also unavoidable even in case of healthy people that their investigated serum, sputum, saliva and from different body fluid (like BAL) contains different kind of fungi nucleic acids from the environment and damaged by the defense mechanism of the healthy immune system (Bolehovska et al., 2006).

The drawbacks of the presently available methods for detecting aspergillus are clearly shown by the fact that these methods are not in clinical use (Cesaro et al., 2008, see e.g. page 2, column 1). A person skilled in the art is well aware of the drawbacks of the presently available methods (see e.g. White et al., Donelly et al., Lewis et al., 2006).

Regarding the previously mentioned facts it can be stated that according to the state of art taking a reliable and unambiguous diagnosis needs the combination of different diagnostic methods which require significant amount of time, energy and financial input. However the screening of the Aspergillosis cases should form a part of preventive routine diagnosis.

Citation or identification of any document in this application is not an admission that such document is available as prior art to the present invention.

### SUMMARY OF THE INVENTION

The object of this invention was to set up a diagnostic method that by itself supplies valid results in a short time (in one day) and by the preventive screening of the high risk patient group gives theoretically a solution for the problem of early diagnosis. Further object of the invention was to set up a diagnostic method which reduces the high number of false positive results caused by cross contaminations.

In some embodiments, an oligonucleotide specific for fungi species of Aspergillus genus which is able to hybridize under stringent conditions to a gene of a fungus species of the Aspergillus genus which may be a homologue of Streptomyces griseus 45H C factor gene. Various embodiments of the homologue gene may include a gene having a sequence identical to any of SEQ ID NOs: 118 to 120. In some embodiments, the oligonucleotide is 12 to 27 nucleotides in length. Various embodiments may include an oligonucleotide having a length of 14 to 25 nucleotides.

Various embodiments may include a pathogenic Aspergillus species as the fungus species of Aspergillus genus. In some embodiments, the oligonucleotide is capable of hybridizing to a gene of a fungus species of Aspergillus genus, for example, a pathogenic Aspergillus species such as Aspergillus fumigatus and/or Aspergillus terreus. In various embodiments, the oligonucleotide has a sequence of any of SEQ ID NOs: 1 to 117 or a functional derivative thereof.

In some embodiments, the oligonucleotide may have a sequence of any of SEQ ID NOs: 1 to 117 or a functional derivative thereof and have a length of 12 to 27 nucleotides.

Some embodiments may include a method for using the oligonucleotide for the detection and/or identification of fungi species of the Aspergillus genus capable of causing aspergillosis. In various embodiments, the method may include hybridizing under stringent conditions the oligonucleotide to a gene or an amplified gene-segment of a fungus species of the Aspergillus genus. The gene or amplified gene segment may be homologous to the Streptomyces griseus 45H C factor gene. In some embodiments the sequence of the homologue gene is identical to any of SEQ ID NOs: 118 to 120.

The method for the identification of Aspergillus fumigates may include an oligonucleotide having a sequence of any of SEQ ID NOs: 1 to 78 or a functional derivative thereof.

In some embodiments, the method for the identification of Aspergillus terreus may include an oligonucleotide having a sequence of any of SEQ ID NOs: 79 to 117 or a functional derivative thereof.

An embodiment of an in vitro diagnostic method for detection and/or identification of fungi species capable of causing aspergillosis may include isolating DNA from a biological sample of a patient. In some embodiments, the sample may include fungi cells. Various embodiments may include a sample which includes blood, tissue, bronchoalveolar lavage and/or sputum. The diagnostic method may include amplifying DNA. In various embodiments, amplifying DNA may include amplifying a gene segment capable of hybridizing under stringent conditions to an oligonucleotide according to any of SEQ ID NOs: 3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114 or 117 in the presence of a fungus species capable of causing aspergillosis. The in vitro diagnostic method may include establishing fungi infection by the identification of the amplified gene segment. In some embodiments, the fungus species causing aspergillosis is Aspergillus fumigatus or Aspergillus terreus.

In various embodiments, PCR or preferably quantitative real-time PCR is performed during amplification of the DNA.

In some embodiments, fluorescent dye or a method based on hydrolysis or hybridization probes is used to identify the amplified gene segment.

Various embodiments may include a diagnostic kit for specific identification of Aspergillus fungi species from biological samples. The kit may include oligonucleotide specific for fungi species of Aspergillus genus or its functional derivative. The oligonucleotide may be able to hybridize under stringent conditions to a gene of a fungus species of the Aspergillus genus which is a homologue of Streptomyces griseus 45H C factor gene. Various embodiments of the homologue gene may include a gene having a sequence identical to any of SEQ ID NOs: 118 to 120. In some embodiments, the oligonucleotide is 14 to 25 nucleotides in length.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 depicts normalized fluorescent values (rn) of Aspergillus fumigatus and A. terreus assays as function of the number of cycles using the indicated amount of template DNA.

FIG. 2 depicts determination of sensitivity of Aspergillus fumigatus TaqMan assay.

FIG. 3 depicts determination of sensitivity of Aspergillus terreus TaqMan assay.

FIG. 4 depicts application of Aspergillus fumigatus specific assay on human genomic template.

### DETAILED DESCRIPTION OF THE INVENTION

Surprisingly present inventors found that the facC gene coding for the extracellular pleiotrop autoregulator protein factor C isolated from Streptomyces griseus 45H (later identified as a member of the species Streptomyces albidoflavus and therefore named Streptomyces albidoflavus 45H), is present in the species Aspergillus fumigatus, Neosartorya fischeri, Aspergillus terreus, Chaetomium globosum and also in Podospora anserina (Biró et al., 1980; Birkó et al., 1999; Kiss et al., 2008). This finding is especially surprising because these fungi species are highly distant relatives of streptomycetes. It was also recognized, that the adequate part of the homologous facC gene is suitable for the exact identification of certain members of Aspergillus genus realizing much less false positive results, than in the case of any so far known methods. The reduction of the number of false positive results may be explained on one hand by the rare presence of the investigated facC gene in fungi, on the other hand by the relative high guanine and cytosine (G+C) content of the gene.

The invention relates to oligonucleotides for the detection of Aspergillus species. The homologues of facC gene of Streptomyces griseus in the species of Aspergillus genus afford the development of oligonucleotide probes that are specific for different Aspergillus species, preferably for pathogenic ones, like A. fumigatus and A. terreus. Hereby the invention offers a better solution than the previously available methods for the diagnosis of infection caused by Aspergillus and for species specific detection of these pathogens.

The invention relates to oligonucleotides which is specific for species in Aspergillus genus, and is able to hybridize under stringent conditions to a gene of a fungus species of the Aspergillus genus, said gene being a homologue of facC gene of Streptomyces griseus 45H. The sequence of said homologous gene is identical to any of SEQ ID NOs 118 to 120.

According to the invention the meaning of the “functional derivative” of an oligonucleotide is an oligonucleotide which contains no more than 1-2 added, substituted, deleted or inserted nucleotides compared to the sequence of the oligonucleotide according to the invention and the G+C ratio of said homologue does not differ with more than 10%, preferably does not differ with more than 5% compared to the oligonucleotide according to the invention, and in addition said homologue is suitable for detection and/or identification of fungi that cause aspergillosis.

The oligonucleotides according to the invention are suitable for the detection and/or identification of fungi preferably Aspergillus fumigatus or Aspergillus terreus that cause aspergillosis.

Since the oligonucleotides according to the invention are not based on ribosomal RNA (rRNA), these oligonucleotides may be used to verify identification methods based on the detection of rRNA. Furthermore if both the method according to the invention based on facC detection and an rRNA based method is performed this combined identification further improves the identification of Aspergillus species.

Additionally the invention relates to an in vitro diagnostic method for detection or identification of fungi species preferably Aspergillus fumigatus or Aspergillus terreus capable of causing aspergillosis, characterized by
a. isolating DNA from a biological sample of a patient, said sample presumably containing fungi cells and wherein preferably said sample is blood, tissue, bronchoalveolar lavage or sputum;
b. amplifying DNA whereby amplifying a gene segment capable of hybridizing under stringent conditions to an oligonucleotide according to any of SEQ ID NOs 3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114 or 117 in the presence of a fungus species capable of causing aspergillosis;
c. establishing fungi infection by the identification of the amplified gene segment.

According to a preferred embodiment of the invention in said method besides the above described gene segment an rRNA segment is also amplified.

According to the invention the term “stringent condition” means such hybridizing and then washing conditions that an ordinary person skilled in the art traditionally considers stringent. In detail hybridization under stringent conditions means that the temperature and the ionic strength is chosen in such a way that hybridization between two complementary DNA fragment is possible. For further definition of stringent conditions see the manual of Sambrook et al. (Sambrook, J. C., Fritsch, E. F. and Maniatis, T., 1989, “Molecular Cloning: A Laboratory Manual”, Cold Spring Harbor Laboratory Press, Cold Spring Harbor, N.Y.). A person skilled in the art will readily recognize that stringent conditions depend on the length (e.g. 10 to 40 base) of DNA sequences, primers, oligonucleotide probes or mixed oligonucleotide probes.

Without limitation a preferred example of stringent conditions is a reaction condition (temperature, composition of the solution etc.) under which the primers and the probe can hybridize with the target sequence only if they show 100% complementarity.

According to an advantageous embodiment of the present invention PCR is performed in step (b), or more preferably real-time quantitative PCR is performed. The real-time quantitative PCR can be performed advantageously with the following oligonucleotides according to the invention:

Aspergillus fumigatus assays in conventional 5′>3′ orientation:

| SEQ ID NO: 1: | |
|---|---|
| CAAAGTCGGCAGCCTTCTG | (19 mer) |
| SEQ ID NO: 2: | |
| TGTCGCGATGCCAAAGGT | (18 mer) |
| SEQ ID NO: 3: | |
| CCGCATTGCTCTGG | (14 mer) |
| SEQ ID NO: 4: | |
| CCTCATCCAAACGCTTCGA | (19 mer) |
| SEQ ID NO: 5: | |
| AGGGCTTTGTGACGGTAGAGATC | (23 mer) |
| SEQ ID NO: 6: | |
| CTCTCTGCCCCCTCC | (15 mer) |
| SEQ ID NO: 7: | |
| GAAACAGCGGGCGACCTAA | (19 mer) |
| SEQ ID NO: 8: | |
| CCGACGTAGTTGCCGTCAA | (19 mer) |
| SEQ ID NO: 9: | |
| ATCACCCAGCTCGAC | (15 mer) |
| SEQ ID NO: 10: | |
| CAGCGGGCGACCTAACAAT | (19 mer) |
| SEQ ID NO: 11: | |
| GGTACATGTGTCCGACGTAGTTG | (23 mer) |
| SEQ ID NO: 12: | |
| CCCAGCTCGACTTT | (14 mer) |
| SEQ ID NO: 13: | |
| CAACTACGTCGGACACATGTACCTA | (25 mer) |
| SEQ ID NO: 14: | |
| TGCGCGCCGAAGGA | (14 mer) |
| SEQ ID NO: 15: | |
| AGAGCTTTGGTCATGGC | (17 mer) |
| SEQ ID NO: 16: | |
| CTCCGCTCAACTGGCAAAG | (19 mer) |
| SEQ ID NO: 17: | |
| TCAATGGCGCAGGTATGCT | (19 mer) |
| SEQ ID NO: 18: | |
| TCAAGCCCGTCGCCGA | (16 mer) |
| SEQ ID NO: 19: | |
| AGCATACCTGCGCCATTGA | (19 mer) |
| SEQ ID NO: 20: | |
| GCTGAGGTGATACCGGACGAT | (21 mer) |
| SEQ ID NO: 21: | |
| CGGTGTACAACCGGCT | (16 mer) |
| SEQ ID NO: 22: | |
| CGGTGTACAACCGGCTGATC | (20 mer) |
| SEQ ID NO: 23: | |
| CATACACGGCGATATGCTTTGA | (22 mer) |
| SEQ ID NO: 24: | |
| TCCGGTATCACCTCAGC | (17 mer) |
| SEQ ID NO: 25: | |
| CAAGCAGCCGGAGTTGGA | (18 mer) |
| SEQ ID NO: 26: | |
| ACTGTCCATACGCTGCATAACC | (22 mer) |
| SEQ ID NO: 27: | |
| ACGCTGTCGTTGACTTT | (17 mer) |
| SEQ ID NO: 28: | |
| GGTTATGCAGCGTATGGACAGTAT | (24 mer) |
| SEQ ID NO: 29: | |
| CCGCTGGCCGCATATG | (16 mer) |
| SEQ ID NO: 30: | |
| ATCTGCTGACGGGAAC | (16 mer) |
| SEQ ID NO: 31: | |
| AATCCCCGACTCTCCACGAT | (20 mer) |
| SEQ ID NO: 32: | |
| TCCGCCAGAGGTCATACGA | (19 mer) |
| SEQ ID NO: 33: | |
| CGACCTCACCAAACC | (15 mer) |
| SEQ ID NO: 34: | |
| GCCCTCGACTCAGGAGACTTG | (21 mer) |
| SEQ ID NO: 35: | |
| CGAGGACCTTTCCGGAGAA | (19 mer) |
| SEQ ID NO: 36: | |
| CAATCAGCCAACTCGA | (16 mer) |
| SEQ ID NO: 37: | |
| CGCCGCGGAACCAAT | (15 mer) |
| SEQ ID NO: 38: | |
| CTGCAGCGTCGGTTTCACT | (19 mer) |
| SEQ ID NO: 39: | |
| AAGCGATACGTATATCTG | (18 mer) |
| SEQ ID NO: 40: | |
| GCCACAATTGACCCCGTTTA | (20 mer) |
| SEQ ID NO: 41: | |
| CACGGTCCCCGTCTGGTAT | (19 mer) |
| SEQ ID NO: 42: | |
| AGCGGTTGATCGTGC | (15 mer) |
| SEQ ID NO: 43: | |
| GCGAGCGCAGGCAATTT | (17 mer) |
| SEQ ID NO: 44: | |
| CCTTGACGAGATGCGGAATC | (20 mer) |
| SEQ ID NO: 45: | |
| TCGAACCCACTGGCC | (15 mer) |
| SEQ ID NO: 46: | |
| AGCGCAGGCAATTTTTCG | (18 mer) |
| SEQ ID NO: 47: | |
| CCTTGACGAGATGCGGAATC | (20 mer) |
| SEQ ID NO: 48: | |
| ACCCACTGGCCAAAT | (15 mer) |
| SEQ ID NO: 49: | |
| TCGAACCCACTGGCCAAAT | (19 mer) |
| SEQ ID NO: 50: | |
| CTTTGCCCCGAGCTCCTT | (18 mer) |
| SEQ ID NO: 51: | |
| AGATTCCGCATCTCGT | (16 mer) |
| SEQ ID NO: 52: | |
| GGACTTGAATACTGGGAAGCTTGT | (24 mer) |
| SEQ ID NO: 53: | |
| CGTCGACCCCGCCTTT | (16 mer) |
| SEQ ID NO: 54: | |
| CAAGGGCCTGTCACC | (15 mer) |
| SEQ ID NO: 55: | |
| GAGCTGAAACTGCCGTTGATG | (21 mer) |
| SEQ ID NO: 56: | |
| CCTCTGCGGTACTGGTTTCG | (20 mer) |
| SEQ ID NO: 57: | |
| AGCCTGTGTACATGGAT | (17 mer) |
| SEQ ID NO: 58: | |
| CGTTGATGCAGCCTGTGTACAT | (22 mer) |
| SEQ ID NO: 59: | |
| GACGCGCGGCTTCCT | (15 mer) |
| SEQ ID NO: 60: | |
| TTCTCGAAACCAGTACCGC | (19 mer) |
| SEQ ID NO: 61: | |
| GATGCAGCCTGTGTACATGGAT | (22 mer) |
| SEQ ID NO: 62: | |
| GACGCGCGGCTTCCT | (15 mer) |
| SEQ ID NO: 63: | |
| TCTCGAAACCAGTACCGCA | (19 mer) |
| SEQ ID NO: 64: | |
| CCTCGTATGGACGCGGTAA | (19 mer) |
| SEQ ID NO: 65: | |
| GACTCCACTATGGCCTGCTAGTC | (23 mer) |
| SEQ ID NO: 66: | |
| TGGGCCACATGTTG | (14 mer) |
| SEQ ID NO: 67: | |
| CCAATACGTCGCATACCTGTCA | (22 mer) |
| SEQ ID NO: 68: | |
| GGCGACCGGCGTATACTTC | (19 mer) |
| SEQ ID NO: 69: | |
| AGACATAGACGACTGCCCT | (19 mer) |
| SEQ ID NO: 70: | |
| AGCGGCGCCTTGGTTAG | (17 mer) |
| SEQ ID NO: 71: | |
| CGACGTCGCAGCCAAAGT | (18 mer) |
| SEQ ID NO: 72: | |
| TTCGCTATGCATATAGACCT | (20 mer) |
| SEQ ID NO: 73: | |
| GTTCCTCGAGCCCCGTTT | (18 mer) |
| SEQ ID NO: 74: | |
| TGTATGGCGGGCATTCG | (17 mer) |
| SEQ ID NO: 75: | |
| CAATTGCAGAAAGTCCCTATA | (21 mer) |
| SEQ ID NO: 76: | |
| TCGAGCCCCGTTTCCAA | (17 mer) |
| SEQ ID NO: 77: | |
| TGTATGGCGGGCATTCG | (17 mer) |
| SEQ ID NO: 78: | |
| TGCAGAAAGTCCCTATATG | (19 mer) |

Aspergillus terreus assays in conventional 5′>3′ orientation:

| SEQ ID NO: 79: | |
|---|---|
| CTCCTCGCATCCAGCGTAAG | (20 mer) |
| SEQ ID NO: 80: | |
| CAGGTCGAATTGGGAAGAAGAC | (22 mer) |
| SEQ ID NO: 81: | |
| TTGGGCGGCGCTAC | (14 mer) |
| SEQ ID NO: 82: | |
| TCTGTGTATCACCCAGCTTGATTT | (24 mer) |
| SEQ ID NO: 83: | |
| AACCCGATCAGGTCCATTGA | (20 mer) |
| SEQ ID NO: 84: | |
| CACGGAAACATTGTCGG | (17 mer) |
| SEQ ID NO: 85: | |
| CGAATGGATACGGGAAGAAGCT | (22 mer) |
| SEQ ID NO: 86: | |
| CGAGCGAGGCATCGGTATG | (19 mer) |
| SEQ ID NO: 87: | |
| TTGCCATTGACAAACTT | (17 mer) |
| SEQ ID NO: 88: | |
| GGCAAAACTCTGTCGCATACC | (21 mer) |
| SEQ ID NO: 89: | |
| GCACCCTCAACAGCAGTGAAT | (21 mer) |
| SEQ ID NO: 90: | |
| ATGCCTCGCTCGCGA | (15 mer) |
| SEQ ID NO: 91: | |
| CTGCTCTATTGACCCGGTGAA | (21 mer) |
| SEQ ID NO: 92: | |
| TTTTTCCCGCTCAGAGCATATC | (22 mer) |
| SEQ ID NO: 93: | |
| ACCGGCTCGTGGTC | (14 mer) |
| SEQ ID NO: 94: | |
| GCATTGCCGTGTTCGATCT | (19 mer) |
| SEQ ID NO: 95: | |
| ATCCGCCAGTTTCGTAGAAAAG | (22 mer) |
| SEQ ID NO: 96: | |
| CGCAACAAAGGGTG | (14 mer) |
| SEQ ID NO: 97: | |
| AACTGGCGGATATCGCTCAT | (20 mer) |
| SEQ ID NO: 98: | |
| AGCATACCCCTGGAACACCTT | (21 mer) |
| SEQ ID NO: 99: | |
| CTTCGCTGGATACGCTAT | (18 mer) |
| SEQ ID NO: 100: | |
| GCTTCTGGTGGTGTGGTCAA | (20 mer) |
| SEQ ID NO: 101: | |
| GACCACCTTCCCAGTATTCAAGTC | (24 mer) |
| SEQ ID NO: 102: | |
| CGCAGGTGACCGCC | (14 mer) |
| SEQ ID NO: 103: | |
| CCGTTTTGAGACAGCGTATGG | (21 mer) |
| SEQ ID NO: 104: | |
| ACGTGGGAGTTGTCGTCACTT | (21 mer) |
| SEQ ID NO: 105: | |
| TACGGAGCGAGCGCT | (15 mer) |
| SEQ ID NO: 106: | |
| TTCGCGTAACGGCACAAG | (18 mer) |
| SEQ ID NO: 107: | |
| GGCGGTCAAAGCATCTTTTC | (20 mer) |
| SEQ ID NO: 108: | |
| ACCCCCGGCGTTGT | (14 mer) |
| SEQ ID NO: 109: | |
| CGCGTAACGGCACAAGCTA | (19 mer) |
| SEQ ID NO: 110: | |
| GGCGGTCAAAGCATCTTTTC | (20 mer) |
| SEQ ID NO: 111: | |
| ACCCCCGGCGTTGT | (14 mer) |
| SEQ ID NO: 112: | |
| TAGACCCCCGGCGTTGTT | (18 mer) |
| SEQ ID NO: 113: | |
| CCTACTCGCTATAGGCGGTCAA | (22 mer) |
| SEQ ID NO: 114: | |
| CCCACTGAAAAGATG | (15 mer) |
| SEQ ID NO: 115: | |
| GATAGAAGAATGCCCTCTCAGCAT | (24 mer) |
| SEQ ID NO: 116: | |
| CGCCAGTGGACGCTCAAC | (18 mer) |
| SEQ ID NO: 117: | |
| CGACGAAGACCACCACA | (17 mer). |

Note: in the grouped sequences of three, the first is the forward primer, the second is the reverse primer and the third is the probe.

In step c) of the above method fluorescent dye or a method based on hydrolysis or hybridisation probes may be used for the identification of the amplified gene-part.

Furthermore the invention relates to a diagnostic kit for the specific identification of Aspergillus fungi from biological samples, which kit contains the oligonucleotide or its functional derivatives according to the invention

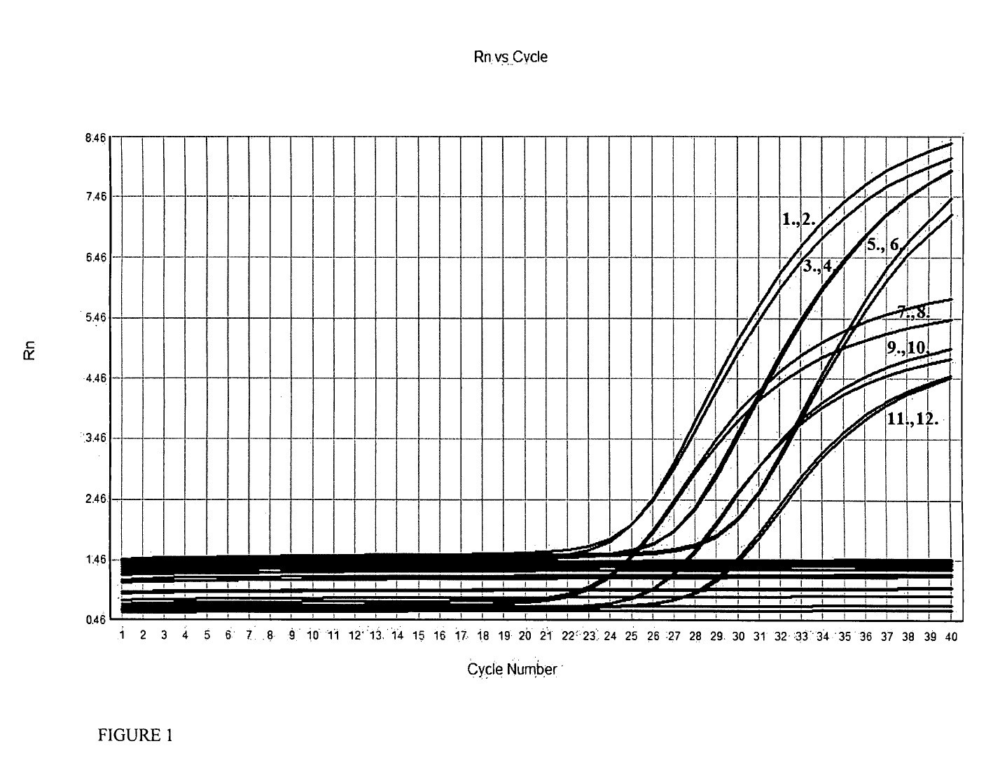

FIG. 1 depicts normalized fluorescent values (rn) of Aspergillus fumigatus and A. terreus assays as function of the number of cycles using the indicated amount of template DNA. In case of using A. fumigatus template and A. fumigatus specific assay curves 1, 2 (4 ng), curves 3, 4 (0.8 ng), and curves 5, 6 (0.16 ng). In case of A. terreus template and A. terreus assay curves 7, 8, 9, 10, and 11, 12 using the same amount of template, respectively, show the amplification. The A. fumigatus TaqMan assay with A. terreus DNA or vice versa gave no amplification.

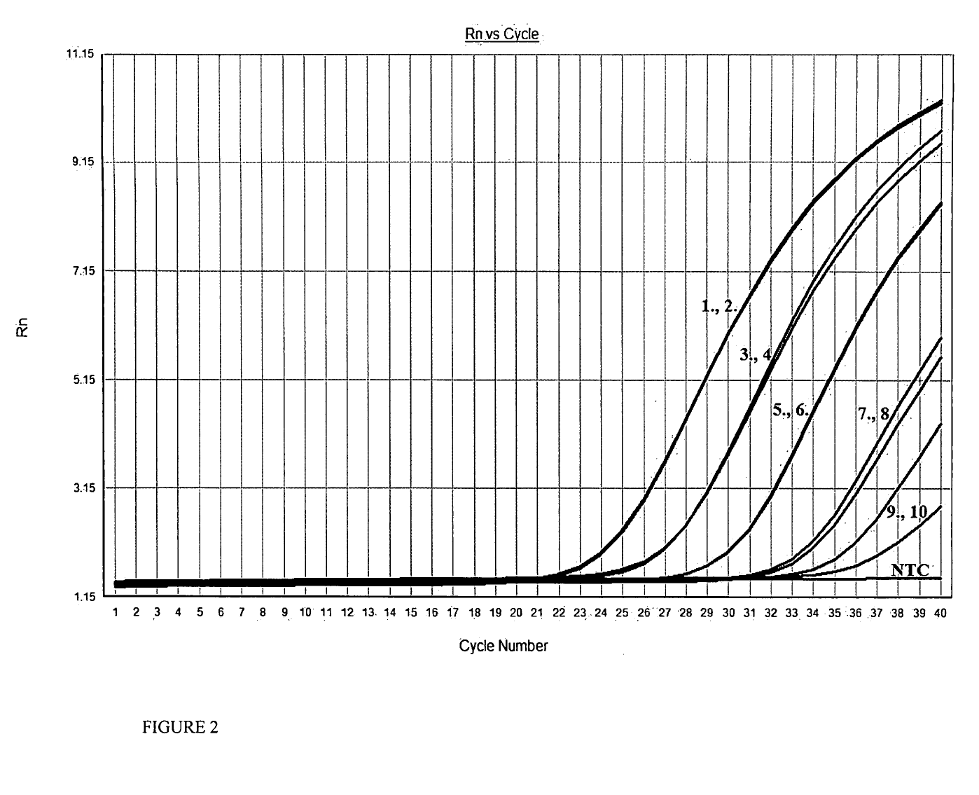

FIG. 2 depicts determination of sensitivity of Aspergillus fumigatus TaqMan assay. Normalised fluorescent values as function of cycle numbers are shown on curves 1, 2 using 4 ng, 3, 4 0.8 ng, 5, 6 0.16 ng, 7, 8 16 pg, and 9, 10 0.16 pg template DNA. NTC (control without template).

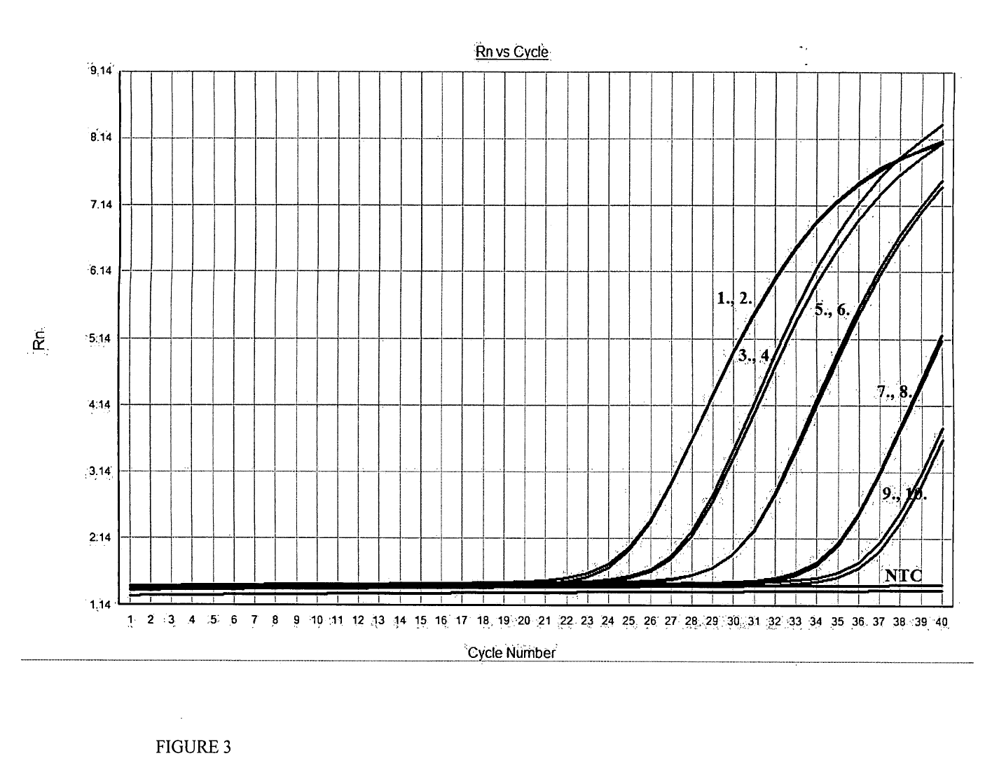

FIG. 3 depicts determination of sensitivity of Aspergillus terreus TaqMan assay. Normalised fluorescent values as function of cycle numbers are showed on curves 1, 2 using 4 ng, 3, 4 0.8 ng, 5, 6 0.16 ng, 7, 8 16 pg, and 9, 10 0.16 pg template DNA. NTC (control without template).

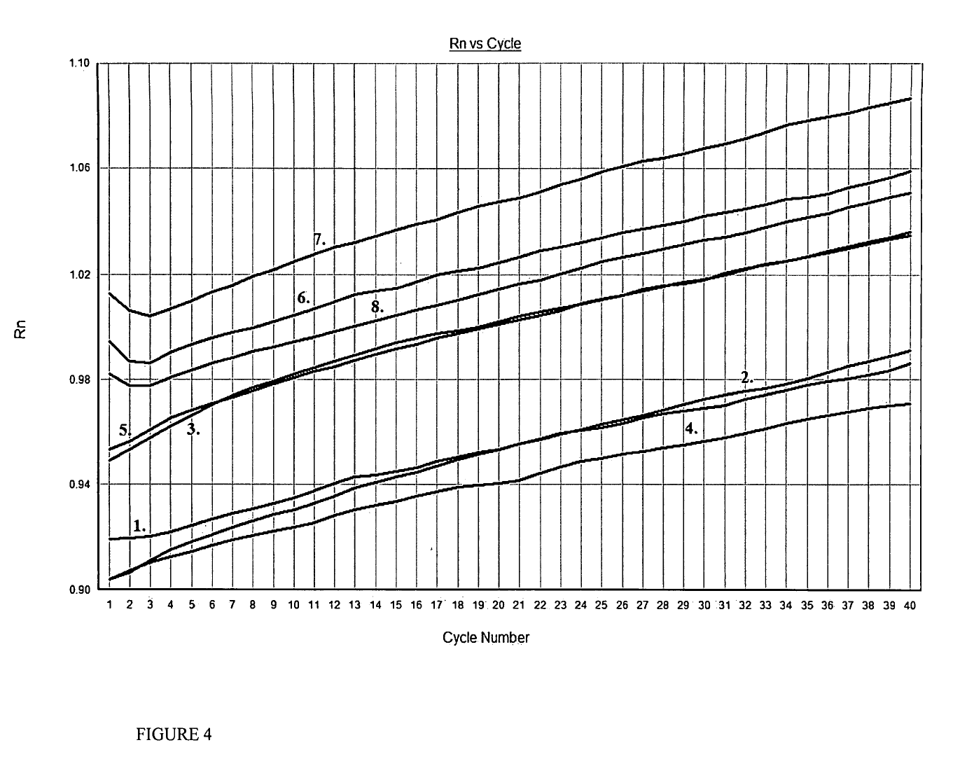

FIG. 4 depicts Application of Aspergillus fumigatus specific assay on human genomic template. DNA originated from three different healthy persons. Curves 1 and 2, 3 and 4, 5 and 6, show the results of the parallel measurements of the same DNA samples. Amount of DNA is 30 ng in each case. Curves 7 and 8 are control without template DNA. Relative fluorescence (rn) does not change significantly as a function of the number of cycles, so human DNA does not give false positive result.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Detection of Aspergillus fumigatus DNA in Biological Sample

According to standard methods described in literature, fungi DNA was isolated from blood, bronchoalveolar lavage, sputum, tissue etc. and DNA concentration was measured.

Measurement was performed in 25 μl final sample volume composed of
12.5 μl TaqMan® Universal PCR Master Mix (2×) (Applied Biosystems part number 4324018).
1.25 μl TaqMan® gene expression assay mix (20×) (Applied Biosystems part number 4332078) containing the forward (SEQ ID NO: 25) and reverse (SEQ ID NO: 26) primers furthermore the FAM (6-carboxilfluoresceine) labeled TaqMan-MGB (“Minor Groove Binder”) probe (SEQ ID NO: 27) that is specific for the investigated gene. Primers and probe were delivered by Applied Biosystems.
2.5 μl tested DNA sample,
8.75 μl nuclease free water.

Measurement was performed by Applied Biosystems 7500 Real Time PCR equipment applying the following reaction parameters:

Part 1: denaturing DNA, 95° C., 10 min.

Part 2: repeating 40× the following steps:
Step 1: 95° C., 15 seconds
Step 2: 60° C., 60 seconds
Step 3: measurement of fluorescence each time after the Step 2.

In case of negative control (NTC, non-template control) 5 μl distilled water was applied instead of the examined sample.

### Example 2

### Detection of Aspergillus terreus DNA in Biological Sample

According to standard methods described in literature, fungi DNA was isolated from blood, bronchoalveolar lavage, sputum, tissue etc. and DNA concentration was established.

Measurement was performed in 25 μl final sample volume composed of:
12.5 μl TaqMan® Universal PCR Master Mix (2×) (Applied Biosystems part number 4324018).
1.25 μl TaqMan® gene expression assay mix (20×) (Applied Biosystems part number 4332078) containing the forward (SEQ ID NO: 85) and reverse (SEQ ID NO: 86) primers furthermore the FAM (6-carboxilfluoresceine) labeled TaqMan-MGB (“Minor Groove Binder”) probe (SEQ ID NO: 87) that is specific for the investigated gene. Primers and probe was delivered by the Applied Biosystems.
2.5 μl tested DNA sample,
8.75 μl nuclease free water.

Measurement was performed by Applied Biosystems 7500 Real Time PCR equipment applying the following reaction parameters:

Part 1: denaturing DNA, 95° C., 10 min.

Part 2: repeating 40× the following steps:
Step 1: 95° C., 15 seconds
Step 2: 60° C., 60 seconds
Step 3: measurement of fluorescence each time after step 2.

In case of negative control (NTC, non-template control) 5 μl distilled water was applied instead of the examined sample.

On the basis of the above described example 1 and 2 it was demonstrated that Aspergillus fumigatus assay gives signal only with Aspergillus fumigatus DNA and not with Aspergillus terreus DNA, furthermore Aspergillus terreus assay gives signal only in case of Aspergillus terreus DNA and not with Aspergillus fumigatus DNA (FIG. 1).

### Example 3

### Determination of the Sensitivity of Aspergillus fumigatus TaqMan Assay

To determine the sensitivity of the assay applying the oligonucleotides according to the invention the method according to example 1 was repeated with different amount of DNA templates. FIG. 2 shows the sensitivity of Aspergillus fumigatus TaqMan assay. The following amounts of template DNA were used: curves 1, 2 applying 4 ng, 3, 4 0.8 ng, 5, 6 0.16 ng, 7, 8 16 pg, and 9, 10 0.16 pg template DNA. According to the figure it can be seen that the sensitivity of the assay is between about 10 and about 200 femtograms, more preferably between about 16 and about 160 femtograms.

### Example 4

### Determination of the Sensitivity of Aspergillus terreus TaqMan Assay

To determine the sensitivity of the measurement methods applying the oligonucleotides described by the invention the method according to example 1 was repeated with different amount of DNA templates. FIG. 3 shows the sensitivity of Aspergillus terreus TaqMan assay. The following amounts of template DNA were used: curves 1 and 2 applying 4 ng, 3, 4 0.8 ng, 5, 6 0.16 ng, 7, 8 16 pg, and 9, 10 0.16 pg template DNA. According to the figure it can be seen that the sensitivity of the assay is between about 10 and about 200 femtograms, more preferably between about 16 and about 160 femtograms.

### Example 5

### Use of Assays Specific for Aspergillus fumigatus and Aspergillus terreus on Human Genomic DNA Template

Assays based on probes according to the invention were investigated for cross-reactions with human DNA. Genomic DNA was isolated from blood samples of three different donors and were used as templates. Neither Aspergillus fumigatus (FIG. 4.) nor Aspergillus terreus assay gave any signal with the human DNA samples. As it can be seen on the figure, the relative fluorescence did not change significantly even during 40 cycles.

The invention is further described by the following numbered paragraphs:

1. An oligonucleotide specific for fungi species of Aspergillus genus which is able to hybridize under stringent conditions to a gene of a fungus species of the Aspergillus genus, said gene being a homologue of Streptomyces griseus 45H C factor gene, wherein the sequence of said homologue gene is identical to any of SEQ ID NO 118 to 120.

2. The oligonucleotide according to claim 1 which is 12 to 27, preferably 14 to 25 nucleotide in length.

3. The oligonucleotide according to claim 1 or 2, wherein the fungi species of Aspergillus genus is a pathogenic Aspergillus species preferably Aspergillus fumigatus or Aspergillus terreus.

4. The oligonucleotide according to any of claims 1 to 3 the sequence of which is any of SEQ ID NO 1 to 117 or a functional derivative thereof.

5. Use of an oligonucleotide according to any of claims 1 to 4 for the detection and/or identification of fungi species capable of causing aspergillosis.

6. Use of an oligonucleotide according to any of claims 1 to 4 for the identification of Aspergillus fumigatus, wherein the sequence of the oligonucleotide is any of SEQ ID NO 1 to 78 or a functional derivative thereof.

7. Use of an oligonucleotide according to any of claims 1 to 4 for the identification of Aspergillus terreus, wherein the sequence of the oligonucleotide is any of SEQ ID NO 79 to 117 or a functional derivative thereof.

8. An in vitro diagnostic method for detection and/or identification of fungi species capable of causing aspergillosis characterized by:

a) isolating DNA isolation from a biological sample of a patient, said sample presumably containing fungi cells and wherein preferably said sample is blood, tissue, bronchoalveolar lavage or sputum;

b) amplifying DNA whereby amplifying a gene segment capable of hybridizing under stringent conditions to an oligonucleotide according to any of SEQ ID NO 3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114 or 117 in the presence of a fungus species capable of causing aspergillosis;

c) establishing fungi infection by the identification of the amplified gene segment.

9. The method according to claim 8, wherein the fungus species causing aspergillosis is Aspergillus fumigatus or Aspergillus terreus

10. The method according to claim 8 or 9, wherein in step (b) PCR or preferably quantitative real-time PCR is performed.

11. The method according to any of claims 8 to 10, wherein in step (c) fluorescent dye or method based on hydrolysis or hybridization probes is used to identify the amplified gene segment.

12. A diagnostic kit for specific identification of Aspergillus fungi species from biological samples, said kit containing an oligonucleotide or its functional derivative according to any of claims 1 to 4.

Having thus described in detail preferred embodiments of the present invention, it is to be understood that the invention defined by the above paragraphs is not to be limited to particular details set forth in the above description as many apparent variations thereof are possible without departing from the spirit or scope of the present invention

### REFERENCES

Abbott S P et al., (1995) Fatal Cerebral Mycoses Caused by the Ascomycete Chaetomium Strumarium. Journal of Clinical Microbiology 10: 2692-2698.
Anandi V et al., (1989) Cerebral Phaeohyphomycosis Caused by Chaetomium globosum in a Renal Transplant Recipient. Journal of Clinical Microbiolog 27 (10): 2226-2229.
Aquino V R et al., (2007) Update on the contribution of galactomannan for the diagnosis of invasive aspergillosis. Mycopathologia 163: 191-202.
Biró S et al., (1980) A substance effecting differentiation in Streptomyces griseus. European Journal of biochemistry/FEBS 103 (2): 359-363.
Birkó Z et al., (1999) Characterization of the gene for factor C, an extracellular signal protein involved in morphological differentiation of Streptomyces griseus. Microbiology 145 (9): 2245-2253
Bolehovska R et al., (2006) Detection of Aspergillus spp. in biological samples by real-time PCR. Biomed Pap Med Fac Univ Palacky Olomouc Czech Repub 150 (2): 245-248.
Cesaro S (2008) Assessment of the lightcycler PCR assay for diagnosis of invasive aspergillosis in paediatric patients with onco-haematological diseases. Mycoses 51 (6):497-504. Epub 2008 Mar. 3.
Denning D W (2006) Aspergillosis. Educational material supplied by The Aspergillus Website. Shering-Plough Corporation.
Donnelly J P (2006) Polymerase chain reaction for diagnosing invasive aspergillosis: getting closer but still a way to go. Clin Infect Dis 42: 487-9.
Erjavec Z and Verweij P E (2002) Recent progress in the diagnosis of fungal infections in the immunocompromised host. Drug Resistance Updates. 5 (1): 3-10.
Florent M et al., (2006) Prospective Evaluation of a Polymerase Chain Reaction-ELISA Targeted to Aspergillus fumigatus and Aspergillus flavus for the Early Diagnosis of Invasive Aspergillosis in Patients with Hematological Malignancies. The Journal of Infectious Diseases 193: 741-7.
Guarro J et al., (2002) Cerebral Aspergillosis Caused by Neosartorya hiratsukae, Brazil. Emerging Infectious Diseases 8 (9):989-91.
Kiss Z et al., (2008) Streptomyces griseus 45H, a producer of the extracellular autoregulator protein factor C, is a member of the species Streptomyces albidoflavus. International Journal of Systematic and Evolutionary Microbiology 58: 1029-1031.
Hummel M et al., (2006) Detection of Aspergillus DNA in Cerebrospinal Fluid from Patients with Cerebral Aspergillosis by a Nested PCR Assay. Journal of Clinical Microbiology. 44 (11): 3989-3993.
Pagano L et al., (2006) The epidemiology of fungal infections in patients with hematologic malignancies: the Sefem-2004 study. Haematologica 91 (8):1068-75.
Sambrook, J. C. et al., (1989) Molecular Cloning: A Laboratory Manual, ed: Cold Spring Harbor Laboratory Press, Cold Spring Harbor, N.Y.
Segal B H and Walsh T J. (2006) Current approaches to diagnosis and treatment of invasive aspergillosis. American Journal of respiratory and critical care medicine 173 (7): 707-17.
Vidal J E et al., (2005) Cerebral Aspergillosis due to Aspergillus fumigatus in AIDS patient: First Culture-proven case reported in Brasil. Revista do Instituto de Medicina Tropical de Säo-Paulo 47 (3): 161-165.
Vonberg, R. P. and Gastmeier, P. (2006) Nosocomial aspergillosis in outbreak settings. Journal of Hospital Infection 63: 246-254.
White P L et al., (2006a) A consensus on fungal polymerase chain reaction diagnosis: a United Kingdom-Ireland evaluation of polymerase chain reaction methods for detection of systemic fungal infections. The Journal of Molecular Diagnosis. 8 (3): 376-384.
White P L et al., (2006b) The Evolution of a Whole Blood Polymerase Chain Reaction Assay for the Detection of Invasive Aspergillosis in Hematology Patients in a Routine Clinical Setting. Clinical Infectious Diseases 42 (4): 487-9.
White P L and Barnes R A (2006) Aspergillus PCR-platforms, strengths and weaknesses. Med Mycol 44: S191-8.
Williamson E C et al., (2000) Aspergillus antigen testing in bone marrow transplant recipients. Journal of Clinical Pathology. 53 (5): 362-366.
Yeghen T et al., (1996) Chaetomium pnemonia in patient with acute myeloid leukaemia. Journal of clinical pathology 49:184-186.

What is claimed is:

1. An oligonucleotide specific for fungi species of Aspergillus genus which is able to hybridize under stringent conditions to a gene of a fungus species of the Aspergillus genus, said gene being a homologue of Streptomyces griseus 45H C factor gene, wherein the sequence of said homologue gene is identical to any of SEQ ID NO: 118 to 120.

2. The oligonucleotide according to claim 1 which is 12 to 27 nucleotides in length.

3. The oligonucleotide according to claim 1, wherein the fungus species of Aspergillus genus is a pathogenic Aspergillus species.

4. The oligonucleotide according to claim 1 the sequence of which is any of SEQ ID NO: 1 to 117 or a functional derivative thereof.

5. A method for using an oligonucleotide according to claim 1 for the detection and/or identification of fungi species of the Aspergillus genus capable of causing aspergillosis, said method comprising hybridizing under stringent conditions said oligonucleotide to a gene or an amplified gene-segment thereof of a fungus species of the Aspergillus genus, said gene being a homologue of Streptomyces griseus 45H C factor gene, wherein the sequence of said homologue gene is identical to any of SEQ ID NO: 118 to 120.

6. The method according to claim 5 for the identification of Aspergillus fumigatus, wherein the sequence of the oligonucleotide is any of SEQ ID NO: 1 to 78 or a functional derivative thereof.

7. The method according to claim 5 for the identification of Aspergillus terreus, wherein the sequence of the oligonucleotide is any of SEQ ID NO: 79 to 117 or a functional derivative thereof.

8. An in vitro diagnostic method for detection and/or identification of fungi species capable of causing aspergillosis characterized by:
a) isolating DNA from a biological sample of a patient, said sample presumably containing fungi cells and wherein preferably said sample is blood, tissue, bronchoalveolar lavage or sputum;
b) amplifying DNA whereby amplifying a gene segment capable of hybridizing under stringent conditions to an oligonucleotide according to any of SEQ ID NO: 3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114 or 117 in the presence of a fungus species capable of causing aspergillosis;
c) establishing fungi infection by the identification of the amplified gene segment.

9. The method according to claim 8, wherein the fungus species causing aspergillosis is Aspergillus fumigatus or Aspergillus terreus.

10. The method according to claim 8, wherein in step (b) PCR or preferably quantitative real-time PCR is performed.

11. The method according to claim 8, wherein in step (c) fluorescent dye or method based on hydrolysis or hybridization probes is used to identify the amplified gene segment.

12. A diagnostic kit for specific identification of Aspergillus fungi species from biological samples, said kit containing an oligonucleotide or its functional derivative according to claim 1.

13. The oligonucleotide according to claim 2 which is 14 to 25 nucleotides in length.

14. The oligonucleotide according to claim 2, wherein the fungus species of Aspergillus genus is a pathogenic Aspergillus species selected from Aspergillus fumigatus or Aspergillus terreus.

15. The oligonucleotide according to claim 14 the sequence of which is any of SEQ ID NO: 1 to 117 or a functional derivative thereof.


SEQUENCE LISTING (120 sequences)

SEQ ID NO: 1 - DNA, 19 bp, Aspergillus fumigatus
caaagtcggcagccttctg

SEQ ID NO: 2 - DNA, 18 bp, Aspergillus fumigatus
tgtcgcgatgccaaaggt

SEQ ID NO: 3 - DNA, 14 bp, Aspergillus fumigatus
ccgcattgctctgg

SEQ ID NO: 4 - DNA, 19 bp, Aspergillus fumigatus
cctcatccaaacgcttcga

SEQ ID NO: 5 - DNA, 23 bp, Aspergillus fumigatus
agggctttgtgacggtagagatc

SEQ ID NO: 6 - DNA, 15 bp, Aspergillus fumigatus
ctctctgccccctcc

SEQ ID NO: 7 - DNA, 19 bp, Aspergillus fumigatus
gaaacagcgggcgacctaa

SEQ ID NO: 8 - DNA, 19 bp, Aspergillus fumigatus
ccgacgtagttgccgtcaa

SEQ ID NO: 9 - DNA, 15 bp, Aspergillus fumigatus
atcacccagctcgac

SEQ ID NO: 10 - DNA, 19 bp, Aspergillus fumigatus
cagcgggcgacctaacaat

SEQ ID NO: 11 - DNA, 23 bp, Aspergillus fumigatus
ggtacatgtgtccgacgtagttg

SEQ ID NO: 12 - DNA, 14 bp, Aspergillus fumigatus
cccagctcgacttt

SEQ ID NO: 13 - DNA, 25 bp, Aspergillus fumigatus
caactacgtcggacacatgtaccta

SEQ ID NO: 14 - DNA, 14 bp, Aspergillus fumigatus
tgcgcgccgaagga

SEQ ID NO: 15 - DNA, 17 bp, Aspergillus fumigatus
agagctttggtcatggc

SEQ ID NO: 16 - DNA, 19 bp, Aspergillus fumigatus
ctccgctcaactggcaaag

SEQ ID NO: 17 - DNA, 19 bp, Aspergillus fumigatus
tcaatggcgcaggtatgct

SEQ ID NO: 18 - DNA, 16 bp, Aspergillus fumigatus
tcaagcccgtcgccga

SEQ ID NO: 19 - DNA, 19 bp, Aspergillus fumigatus
agcatacctgcgccattga

SEQ ID NO: 20 - DNA, 21 bp, Aspergillus fumigatus
gctgaggtgataccggacgat

SEQ ID NO: 21 - DNA, 16 bp, Aspergillus fumigatus
cggtgtacaaccggct

SEQ ID NO: 22 - DNA, 20 bp, Aspergillus fumigatus
cggtgtacaaccggctgatc

SEQ ID NO: 23 - DNA, 22 bp, Aspergillus fumigatus
catacacggcgatatgctttga

SEQ ID NO: 24 - DNA, 17 bp, Aspergillus fumigatus
tccggtatcacctcagc

SEQ ID NO: 25 - DNA, 18 bp, Aspergillus fumigatus
caagcagccggagttgga

SEQ ID NO: 26 - DNA, 22 bp, Aspergillus fumigatus
actgtccatacgctgcataacc

SEQ ID NO: 27 - DNA, 17 bp, Aspergillus fumigatus
acgctgtcgttgacttt

SEQ ID NO: 28 - DNA, 24 bp, Aspergillus fumigatus
ggttatgcagcgtatggacagtat

SEQ ID NO: 29 - DNA, 16 bp, Aspergillus fumigatus
ccgctggccgcatatg

SEQ ID NO: 30 - DNA, 16 bp, Aspergillus fumigatus
atctgctgacgggaac

SEQ ID NO: 31 - DNA, 20 bp, Aspergillus fumigatus
aatccccgactctccacgat

SEQ ID NO: 32 - DNA, 19 bp, Aspergillus fumigatus
tccgccagaggtcatacga

SEQ ID NO: 33 - DNA, 15 bp, Aspergillus fumigatus
cgacctcaccaaacc

SEQ ID NO: 34 - DNA, 21 bp, Aspergillus fumigatus
gccctcgactcaggagacttg

SEQ ID NO: 35 - DNA, 19 bp, Aspergillus fumigatus
cgaggacctttccggagaa

SEQ ID NO: 36 - DNA, 16 bp, Aspergillus fumigatus
caatcagccaactcga

SEQ ID NO: 37 - DNA, 15 bp, Aspergillus fumigatus
cgccgcggaaccaat

SEQ ID NO: 38 - DNA, 19 bp, Aspergillus fumigatus
ctgcagcgtcggtttcact

SEQ ID NO: 39 - DNA, 18 bp, Aspergillus fumigatus
aagcgatacgtatatctg

SEQ ID NO: 40 - DNA, 20 bp, Aspergillus fumigatus
gccacaattgaccccgttta

SEQ ID NO: 41 - DNA, 19 bp, Aspergillus fumigatus
cacggtccccgtctggtat

SEQ ID NO: 42 - DNA, 15 bp, Aspergillus fumigatus
agcggttgatcgtgc

SEQ ID NO: 43 - DNA, 17 bp, Aspergillus fumigatus
gcgagcgcaggcaattt

SEQ ID NO: 44 - DNA, 20 bp, Aspergillus fumigatus
ccttgacgagatgcggaatc

SEQ ID NO: 45 - DNA, 15 bp, Aspergillus fumigatus
tcgaacccactggcc

SEQ ID NO: 46 - DNA, 18 bp, Aspergillus fumigatus
agcgcaggcaatttttcg

SEQ ID NO: 47 - DNA, 20 bp, Aspergillus fumigatus
ccttgacgagatgcggaatc

SEQ ID NO: 48 - DNA, 15 bp, Aspergillus fumigatus
acccactggccaaat

SEQ ID NO: 49 - DNA, 19 bp, Aspergillus fumigatus
tcgaacccactggccaaat

SEQ ID NO: 50 - DNA, 18 bp, Aspergillus fumigatus
ctttgccccgagctcctt

SEQ ID NO: 51 - DNA, 16 bp, Aspergillus fumigatus
agattccgcatctcgt

SEQ ID NO: 52 - DNA, 24 bp, Aspergillus fumigatus
ggacttgaatactgggaagcttgt

SEQ ID NO: 53 - DNA, 16 bp, Aspergillus fumigatus
cgtcgaccccgccttt

SEQ ID NO: 54 - DNA, 15 bp, Aspergillus fumigatus
caagggcctgtcacc

SEQ ID NO: 55 - DNA, 21 bp, Aspergillus fumigatus
gagctgaaactgccgttgatg

SEQ ID NO: 56 - DNA, 20 bp, Aspergillus fumigatus
cctctgcggtactggtttcg

SEQ ID NO: 57 - DNA, 17 bp, Aspergillus fumigatus
agcctgtgtacatggat

SEQ ID NO: 58 - DNA, 22 bp, Aspergillus fumigatus
cgttgatgcagcctgtgtacat

SEQ ID NO: 59 - DNA, 15 bp, Aspergillus fumigatus
gacgcgcggcttcct

SEQ ID NO: 60 - DNA, 19 bp, Aspergillus fumigatus
ttctcgaaaccagtaccgc

SEQ ID NO: 61 - DNA, 22 bp, Aspergillus fumigatus
gatgcagcctgtgtacatggat

SEQ ID NO: 62 - DNA, 15 bp, Aspergillus fumigatus
gacgcgcggcttcct

SEQ ID NO: 63 - DNA, 19 bp, Aspergillus fumigatus
tctcgaaaccagtaccgca

SEQ ID NO: 64 - DNA, 19 bp, Aspergillus fumigatus
cctcgtatggacgcggtaa

SEQ ID NO: 65 - DNA, 23 bp, Aspergillus fumigatus
gactccactatggcctgctagtc

SEQ ID NO: 66 - DNA, 14 bp, Aspergillus fumigatus
tgggccacatgttg

SEQ ID NO: 67 - DNA, 22 bp, Aspergillus fumigatus
ccaatacgtcgcatacctgtca

SEQ ID NO: 68 - DNA, 19 bp, Aspergillus fumigatus
ggcgaccggcgtatacttc

SEQ ID NO: 69 - DNA, 19 bp, Aspergillus fumigatus
agacatagacgactgccct

SEQ ID NO: 70 - DNA, 17 bp, Aspergillus fumigatus
agcggcgccttggttag

SEQ ID NO: 71 - DNA, 18 bp, Aspergillus fumigatus
cgacgtcgcagccaaagt

SEQ ID NO: 72 - DNA, 20 bp, Aspergillus fumigatus
ttcgctatgcatatagacct

SEQ ID NO: 73 - DNA, 18 bp, Aspergillus fumigatus
gttcctcgagccccgttt

SEQ ID NO: 74 - DNA, 17 bp, Aspergillus fumigatus
tgtatggcgggcattcg

SEQ ID NO: 75 - DNA, 21 bp, Aspergillus fumigatus
caattgcagaaagtccctata

SEQ ID NO: 76 - DNA, 17 bp, Aspergillus fumigatus
tcgagccccgtttccaa

SEQ ID NO: 77 - DNA, 17 bp, Aspergillus fumigatus
tgtatggcgggcattcg

SEQ ID NO: 78 - DNA, 19 bp, Aspergillus fumigatus
tgcagaaagtccctatatg

SEQ ID NO: 79 - DNA, 20 bp, Aspergillus terreus
ctcctcgcatccagcgtaag

SEQ ID NO: 80 - DNA, 22 bp, Aspergillus terreus
caggtcgaattgggaagaagac

SEQ ID NO: 81 - DNA, 14 bp, Aspergillus terreus
ttgggcggcgctac

SEQ ID NO: 82 - DNA, 24 bp, Aspergillus terreus
tctgtgtatcacccagcttgattt

SEQ ID NO: 83 - DNA, 20 bp, Aspergillus terreus
aacccgatcaggtccattga

SEQ ID NO: 84 - DNA, 17 bp, Aspergillus terreus
cacggaaacattgtcgg

SEQ ID NO: 85 - DNA, 22 bp, Aspergillus terreus
cgaatggatacgggaagaagct

SEQ ID NO: 86 - DNA, 19 bp, Aspergillus terreus
cgagcgaggcatcggtatg

SEQ ID NO: 87 - DNA, 17 bp, Aspergillus terreus
ttgccattgacaaactt

SEQ ID NO: 88 - DNA, 21 bp, Aspergillus terreus
ggcaaaactctgtcgcatacc

SEQ ID NO: 89 - DNA, 21 bp, Aspergillus terreus
gcaccctcaacagcagtgaat

SEQ ID NO: 90 - DNA, 15 bp, Aspergillus terreus
atgcctcgctcgcga

SEQ ID NO: 91 - DNA, 21 bp, Aspergillus terreus
ctgctctattgacccggtgaa

SEQ ID NO: 92 - DNA, 22 bp, Aspergillus terreus
tttttcccgctcagagcatatc

SEQ ID NO: 93 - DNA, 14 bp, Aspergillus terreus
accggctcgtggtc

SEQ ID NO: 94 - DNA, 19 bp, Aspergillus terreus
gcattgccgtgttcgatct

SEQ ID NO: 95 - DNA, 22 bp, Aspergillus terreus
atccgccagtttcgtagaaaag

SEQ ID NO: 96 - DNA, 14 bp, Aspergillus terreus
cgcaacaaagggtg

SEQ ID NO: 97 - DNA, 20 bp, Aspergillus terreus
aactggcggatatcgctcat

SEQ ID NO: 98 - DNA, 21 bp, Aspergillus terreus
agcatacccctggaacacctt

SEQ ID NO: 99 - DNA, 18 bp, Aspergillus terreus
cttcgctggatacgctat

SEQ ID NO: 100 - DNA, 20 bp, Aspergillus terreus
gcttctggtggtgtggtcaa

SEQ ID NO: 101 - DNA, 24 bp, Aspergillus terreus
gaccaccttcccagtattcaagtc

SEQ ID NO: 102 - DNA, 14 bp, Aspergillus terreus
cgcaggtgaccgcc

SEQ ID NO: 103 - DNA, 21 bp, Aspergillus terreus
ccgttttgagacagcgtatgg

SEQ ID NO: 104 - DNA, 21 bp, Aspergillus terreus
acgtgggagttgtcgtcactt

SEQ ID NO: 105 - DNA, 15 bp, Aspergillus terreus
tacggagcgagcgct

SEQ ID NO: 106 - DNA, 18 bp, Aspergillus terreus
ttcgcgtaacggcacaag

SEQ ID NO: 107 - DNA, 20 bp, Aspergillus terreus
ggcggtcaaagcatcttttc

SEQ ID NO: 108 - DNA, 14 bp, Aspergillus terreus
acccccggcgttgt

SEQ ID NO: 109 - DNA, 19 bp, Aspergillus terreus
cgcgtaacggcacaagcta

SEQ ID NO: 110 - DNA, 20 bp, Aspergillus terreus
ggcggtcaaagcatcttttc

SEQ ID NO: 111 - DNA, 14 bp, Aspergillus terreus
acccccggcgttgt

SEQ ID NO: 112 - DNA, 18 bp, Aspergillus terreus
tagacccccggcgttgtt

SEQ ID NO: 113 - DNA, 22 bp, Aspergillus terreus
cctactcgctataggcggtcaa

SEQ ID NO: 114 - DNA, 15 bp, Aspergillus terreus
cccactgaaaagatg

SEQ ID NO: 115 - DNA, 24 bp, Aspergillus terreus
gatagaagaatgccctctcagcat

SEQ ID NO: 116 - DNA, 18 bp, Aspergillus terreus
cgccagtggacgctcaac

SEQ ID NO: 117 - DNA, 17 bp, Aspergillus terreus
cgacgaagaccaccaca

SEQ ID NO: 118 - DNA, 948 bp, Aspergillus fumigatus
atgactccgctcgtcaaagtcggcagccttctggccgcattgctctgggtcacctttggcatcgcgacagtcagcgccagcatcccctcatccaaacgcttcgatctctctgccccctccgccgatctctaccgtcacaaagccctccgcgatgacacagtccagcaaggattcgcctttgacaatgtcaaccgccgccttttcgtcgcccagcgccgcgacggctcctccgaaacagcgggcgacctaacaatcacccagctcgactttgacggcaactacgtcggacacatgtacctaaagagctttggtcatggcgtctccttcggcgcgcaggccgttgggtccgcaacctacctctggaccgaggtcgacgccaacgccaacggctacggcaagcaactcgcccgcttcaagttcgcctcgggcaccacgctgaccagctcctccgctcaactggcaaagttcaagcccgtcgccgacgcaacggagcatacctgcgccattgacccggtgtacaaccggctgatcgtccggtatcacctcagcgggtcaaagcatatcgccgtgtatgatcttgctgcggcgacgcgcggcgatttctccgccccgctggcgaacttcaagcagccggagttggagacgctgtcgttgactttccagggttatgcagcgtatggacagtatctgtatctgctgacgggaacttcatatgcggccagcggaggtaaagtcaattccgaggtaaccagtgttgacatgaacacgggcaaggtggtgcaggggcccacgctcaccaaggccggttccacgctggagtttagagagccggagggattagcgatttacaagacggctgcggggcaggtgaggttgttcttggggtttgcttcggggaaggcgggtgatcgcaggtcgaatctgttctacaagaatgtgttggtttag

SEQ ID NO: 119 - DNA, 933 bp, Aspergillus fumigatus
atgcgagtctccctctttgctatcatcacagcactaacatgtgtgattgccaaaatccccgactctccacgattcgacctcaccaaaccgtcgtatgacctctggcggaacaagaaaaccgccgccaaaaccgtccaacagtcattcacctttgacaacgtcaaccgccgcctgttcatcgtaaaccgtcgcgatggctccgccctcgactcaggagacttgacaatcagccaactcgacttctccggaaaggtcctcggctcaatgcacctgctcggctgcggccacggtgtgaacatcgccgcggaaccaatcggaagcgatacgtatatctggagtgaaaccgacgctgcagccagcggatacgggacagcattgtggaagttcaagttccagaatggcaagacgttgaatagcgccacggataagcgcaagcggattgtgccgttgcccgagtttgacaggggcactgccacaattgaccccgtttataagcggttgatcgtgcgataccagacggggaccgtgcagaacatcgccgcgtttgaccttgctgaggcgagcgcaggcaatttttcgaacccactggccaaatgggagattccgcatctcgtcaaggagctcggggcaaaggttaacgtctttcagggatatacagcttacgggcggtatgtctatttcctgactggggaatcttatgacgctagtggtggtaagctgaattcagaggttatggctctggacttgaatactgggaagcttgtgcaagggcctgtcaccacaaaggcggggtcgacgttgaaatttcgtgagccggaggggatggcaatctataagacggccagtggggaggtgaggcttttcttgggattcgcgtcgggggttgcaggagaccggaggagtaatctgttttacaagaacgcgctgatatag

SEQ ID NO: 120 - DNA, 387 bp, Aspergillus terreus
atgcgtcctttcctccaaactattgtactcctcgcatccagcgtaagcctggcttgggcggcgctaccgtcttcttcccaattcgacctgtcagcgccatcttacgacctcttccgcagcaagccccttgctgacgcgacagtccaacaaggcttctccttcgacaatgtcaatcatcgcctctttgtcgcccaacgccgcgacgggtcctccgaaaccgccggcgatctgtgtatcacccagcttgatttccacggaaacattgtcggatcaatggacctgatcgggtttggccacggcgtgtcctttgccgctcagccagtcgtttgtcaatggcaaaactctgtcgcataccgatgcctcgctcgcgaaattcactgctgttga